(19) United States
(12) Patent Application Publication
(10) Pub. No.: US 2020/0164111 A1
(43) Pub. Date: May 28, 2020

(54) IMPLANT FOR SUBCUTANEOUS IMPLANTATION

(71) Applicants: Christian Kasperk, Heidelberg (DE); Andreas Haas, Sandhausen (DE)

(72) Inventor: Andreas Haas, Sandhausen (DE)

(73) Assignees: Christian Kasperk, Heidelberg (DE); Andreas Haas, Sandhausen (DE)

(21) Appl. No.: 16/668,529

(22) Filed: Oct. 30, 2019

(30) Foreign Application Priority Data
Oct. 31, 2018 (DE) 102018127200.6

(51) Int. Cl. A61L 27/54; A61L 27/58; A61L 27/26; C08L 33/06

(52) U.S. Cl. CPC A61L 27/54; A61L 27/58; A61L 27/26; C08L 33/06; C08L 2205/025; A61L 2300/204; A61L 2300/604; A61L 2300/43; A61L 2300/412; A61L 2300/222

(57) ABSTRACT

The invention relates to an implant for subcutaneous implantation having a substantially cylindrical, conical or spiral-shaped base body which has a lateral surface. The base body consists of a completely resorbable biodegradable polymer. The implant is further configured for blunt implantation. In addition, the biodegradable polymer is enriched with a medicinally relevant active ingredient.

The invention relates to an implant for subcutaneous implantation of a drug carrier and in particular to an implant for use in the treatment of diseases which require a continuous therapy.

Examples thereof are the treatment of syndromes associated with estrogen deficiency in postmenopausal women, gynecologically or endocrinologically indicated permanent estrogen treatment of women of any age, for example also male-to-female transsexuality, or also the treatment of androgenetic hair loss, especially in men. Since permanent estrogen treatment in women is an established routine in endocrinological and gynecological practice, only the treatment and backgrounds of a pharmacological permanent treatment of androgenetic alopecia using a drug carrier implant having novel technical properties will be discussed hereinbelow in greater detail by way of example.

Androgenetic alopecia is a genetically predisposed occurrence in adulthood, in particular in men. It leads to irreversible hair loss of the hair of the head. It is caused by the hair roots shriveling and vellus hair follicles developing. This is explained by a change in the dynamics in the hair cycle, which then leads to vellus transformation of the hair follicle.

The hair growth cycle, which is also referred to as the hair cycle, is divided essentially into three phases, an anagen phase, a catagen phase and a telogen phase. The anagen phase is an active growth phase, which can last from two years up to about six years. In this phase, a new hair follicle forms, which causes a hair to be generated. In this phase, the hair accordingly grows for many years and increases in length.

There then follows a short stage, which is known as the catagen phase and lasts from one to two weeks. In this stage, the hair follicle shortens, and the hair is detached.

This is then followed by the resting phase, known as the telogen phase, which lasts from five to six weeks up to about 100 days. In this phase, the hair follicle regenerates so that the next anagen phase and thus the formation of a new hair can follow.

In androgenetic alopecia, the equilibrium of the various phases is shifted among each other. Thus, the duration of the anagen phase gradually becomes shorter while the duration of the telogen phase increases. Since the duration of the anagen phase determines the length of the hair, the maximum length of the new anagen hair becomes shorter than that of its predecessor. Ultimately, after several cycles, this leads to miniaturization culminating in shriveling of the hair follicle and to a hairless appearance (balding).

A fundamental role in maintaining hair growth has in the meantime been attributed to the papillae of the skin. Communication between the papillae of the skin and the hair follicle cells, which unfolds under the influence of androgens, such as the hormone testosterone, results from the secretion of cytokines and growth factors from the papillae. These factors have an autocrine effect on the dermal papillae themselves and a paracrine effect on the hair follicle epithelial cells. Testosterone, as a steroid hormone, can penetrate freely into the cell membrane and is converted in the cytoplasm into dihydrotestosterone (DHT) by 5-α-reductase, mainly of type II. DHT binds much more strongly to the androgen receptor (AR) than does testosterone, and this complex, supported by the AR coactivators, is transported into the cell nucleus. This leads in the cell nucleus to target gene transcription and then to the translation of the above-mentioned factors, which then exert biological activity on the papilla of the skin and also on the hair follicle and influence the hair follicle cycle.

These factors include inter alia the insulin like growth factor (IGF-1), the basic fibroblast factor (bFGF), the vascular endothelial growth factor (VEGF), cytokines such as transforming growth factor beta 1 (TGFβ 1), interleukin 1 alpha (IL-1α) and tumor necrosis factor alpha (TNF α). Many studies have described the role of androgens as well as the paracrine interactions between the papillae of the skin and the hair follicles as being a crucial process in the miniaturization and shriveling of hair follicles. The higher the concentration of androgens and androgen receptors, the greater the effect on the expression of genes which disrupt the hair follicle cycle. This leads to premature termination of the anagen phase, which is associated with premature entry into the catagen phase. The catagen phase occurs as a consequence of the reduced expression of the factors that sustain the anagen phase, such as the growth factors IGF-1, bFGF and VEGF. In addition, increased expression of cytokines (TGFβ1, IL-1α and TNFα) promotes apoptosis, that is to say the death of the cells of the hair follicle.

It has recently been reported that the peptide DKK-1 is upregulated by the androgen DHT, which leads to inhibition of the outer root sheath cells of the hair follicle and ultimately to induction of apoptosis of those cells.

One therapeutic approach consists in inhibiting the conversion of the natural androgen testosterone into dihydrotestosterone (DHT), which acts more strongly locally on the androgen receptor, by blocking the enzyme 5-α-reductase, so that the expression of the factors that adversely affect the hair follicle cycle is inhibited. The active ingredients finasteride and dutasteride are known in this context.

Studies of the administration of 1 mg of finasteride per day, or also of the administration of from 2 mg to 10 mg of minoxidil per day, show an inhibiting action of these substances on the development of alopecia, provided that the substances are taken daily and for life. These studies show success in the sense of ending hair loss and in some cases renewed growth of hairs.

The substance dutasteride is also a competitive inhibitor of “type 1” and “type 2” 5-α-reductase and likewise shows favorable effects on the prevention of androgenetic alopecia when taken daily in a dose of 0.5 mg.

Reassuringly, the long-term administration of 5-α-reductase inhibitors is not associated with a disadvantageous effect on muscle metabolism, since the anabolic effect of testosterone on muscle is independent of the presence of dihydrotestosterone. Sexual side-effects, which are always feared, of the long-term use of 5-α-reductase inhibitors also tend to be uncommon and have at most mild clinical symptoms and, according to the studies available at present, are reversible when the 5-α-reductase inhibitors are discontinued.

The topical administration of the known antihypertensive minoxidil, a blood pressure reducer, also leads to increased hair growth as a side-effect. The mechanism of action thereby has not yet been clarified. It is supposed that minoxidil, by widening the capillaries, promotes local blood flow at the hair follicle. It is further supposed that the telogen phase of the hair cycle is shortened, so that the growth phase (anagen phase) begins more quickly.

However, a fundamental requirement for the success of these existing therapies is that these substances are taken extremely regularly, and consequently daily and for life. All the described substances used hitherto for preventing androgenetic alopecia can be used effectively only if they are taken on the one hand in good time, at the first signs of increased hair loss as part of the hormonal changes which occur in early adulthood, and on the other hand, consequently, also for life. In good time here means at the first signs of increased hair loss as part of the hormonal changes which occur in early adulthood. However, daily oral intake or daily topical administration is onerous.

It is a problem with all these forms of therapy, therefore, that, if the treatment is to be successful, an extremely continuous, ideally daily, intake or administration is necessary, otherwise the success of the therapy cannot be guaranteed. A further problem is that, in particular in the case of topical administration, third persons may also come into contact with the active ingredient. Depending on the active ingredient used, there is the risk here that women in particular may come into contact therewith, whereby simple skin contact, for example, is already sufficient. In pregnant women, finasteride causes embryonic abnormalities and the like. Cycle disorders are also known.

Similar problems also exist in the therapy of other disorders using different active ingredients. This concerns in particular therapies using active ingredients which require an onerous daily intake in order to achieve a desired drug level and thus extraordinary patient compliance, but also therapies using active ingredients which can be toxic for fellow humans with whom one comes into direct contact and which could then unintentionally be absorbed dermally by the other person or partner.

The object underlying the invention is, therefore, to provide an implant which is suitable for the continuous administration of active ingredients but causes minimal outlay on implantation.

The object is achieved according to the invention by an implant for subcutaneous implantation having the features of claim 1.

Advantageous embodiments of the invention are given in the dependent claims.

According to the invention, an implant for subcutaneous implantation has a substantially cylindrical, conical or spiral-shaped base body which has a lateral surface, wherein the base body consists of a completely resorbable biodegradable polymer. It is further provided that the implant is configured for blunt implantation and there are provided on the lateral surface of the base body, in the case of a cylindrical or conical base body, protruding wings which are integrally formed with the base body. The wings are made of a similar or the same material as the base body. It is further provided that the biodegradable polymer is enriched or supplemented with at least one medicinally relevant active ingredient.

In the case of a spiral-shaped base body, the wings can, but do not have to be, omitted. This design can be similar to a corkscrew or a spring. The cross-section of the base body can be round or polygonal.

A fundamental idea of the invention can be seen in the provision of an implant which is implanted into a body and can be left there. Furthermore, this implant is completely resorbable owing to the use of biodegradable polymers. This means that the implant must only be implanted into the body once, preferably subcutaneously, and decomposes or is degraded during the duration of action. A further intervention to remove the implant is thus prevented from being required. It is further provided according to the invention that the implant is configured for blunt implantation. It is also important that, owing to the shaping, the implant is prevented from slipping out, or at least it is more difficult for it to slip out.

Within the meaning of the invention, blunt implantation is to be understood as meaning that the step of creating an implant bed can be omitted and/or implantation takes place substantially atraumatically. This means that, when the implant is introduced into a body, the tissue of the body is substantially displaced and a cavity for receiving the implant does not actively have to be created.

Finally, it has been recognized according to the invention that the biodegradable polymer can be enriched with one or more medicinally relevant active ingredients, that is to say a pharmacological substance. Owing to the continuous degradation of the polymer, the active ingredient or ingredients is/are continuously distributed or released, so that the active ingredient or ingredients can be administered continuously over a specific period of time and its or their systemic effects can unfold.

It is advantageous if the wings are arranged around the entire periphery of the lateral surface and, in a radial cross-section of the implant, have a surface substantially similar to a triangle. The wings can also be curved, for example. The wings can also be configured in the manner of a spiral, whereby they form at least one screw thread. Another possibility is to provide the base body itself in the form of a spiral. A further form according to the invention is to provide the wings in the form of barbed hooks, which prevent the implant from being pulled out or, in particular, from slipping out by itself.

It is fundamental to all possible wing forms that they permit, or assist with, blunt introduction of the implant into a body but ideally prevent the implant from being pulled back, being pushed back and/or slipping back. In one embodiment, they can also be configured to be similar to barbed hooks which prevent the implant from being pulled back, being pushed back and/or slipping back.

In an advantageous embodiment, the implant is configured to be self-inserting and/or self-tapping on rotation about the longitudinal axis of the base body. In this respect, the implant can be regarded as being a screw-in implant which, similarly to a self-tapping screw, namely by rotation about the longitudinal axis of the base body, draws itself into the body. This in turn has the advantage that it is possible to dispense with the complex process of creating an implant bed with the corresponding problems on enlargement of the cavity, for example bleeding, pain, infection.

For example, the implant according to the invention can thus be implanted subcutaneously at a clinically safer, less disruptive and sufficiently voluminous site, for example in the stomach region. For this purpose, a local anesthetic may be required. Implantation takes place, for example, with a minimal stab incision, through which the implant is then screwed into, for example, the subcutaneous tissue in a self-inserting manner. As described, the necessary steps of creating the implant bed by means of a specially configured cavity enlarger are thus omitted.

Furthermore, introduction of the implant provided with barbed hooks can take place also only to push in, similarly to the introduction of a wall plug, only the direction of the interior of the body, whereby the implant is secured against slipping back.

In principle, the implant can have any desired resorption rate. Preferably, it is at least three months. Within the meaning of the invention, resorption rate is understood as meaning the time taken by a body into which the implant has been inserted to degrade, that is to say reabsorb, the implant completely. It is preferred if this period of time is as long as possible, for example two or three years. There may be used as components of the biodegradable polymer, for example, polylactides, polyglycoids, polylactide-co-glycolides, polycaprolactans or oxalate-crosslinked oligocaprolactans.

Release of the active ingredients present takes place by polymer erosion, whereby a distinction is substantially made between two forms, which can also occur simultaneously. On the one hand, surface erosion, in which the biodegradable polymer is degraded from the outside in, and on the other hand bulk erosion, in which substantially all the polymer molecules are degraded simultaneously.

General known factors which influence the degradation are the pH, the particle size and the diffusion of water into the material. Biodegradable polymers are degraded in aqueous media to monomers. The monomers are metabolized and expelled via the lungs in the form of water and carbon dioxide. A further portion, which in most cases is relatively small, can also be excreted via the urine.

If the chosen biodegradable polymer is composed of several different polymers, for example polylactide and polyglycoid, then the rate of degradation can be influenced by the ratio of the two materials to one another. One property which contributes substantially to the variation in the rate of degradation is the hydrophilicity of the polymers used, since the amount of water taken up into the implant material can thus be influenced, and hence the rate of degradation is influenced. Similarly, the biodegradable polymer can be composed of polycaprolactan and oxalate-crosslinked oligocaprolactan, whereby the ratio of the two components to one another again determines the rate of degradation.

It has been found to be advantageous if the implant does not have a sharp tip but a tip that is at least slightly rounded or somewhat blunt. In other words, the tip is a tissue-conserving, rounded tip. This contributes to allowing the blunt implantation within the meaning of the invention. In this manner, tissue damage on introduction of the implant is prevented or reduced because, as already described, the tissue is substantially not cut but merely displaced. Less damage is thus caused to the tissue, which leads to better compatibility.

In principle, any desired active ingredients having medicinal activity can be introduced into the implant. For the therapy of androgenetic alopecia there are suitable, for example, a competitive inhibitor of type 1 and/or type 2 5-α-reductase, in particular dutasteride, finasteride and/or the substance minoxidil. As already described at the beginning, it is known that these substances can retard and in the best case even stop hair loss. When the implant according to the invention is used, the possibility of constant delivery of the active ingredient over a prolonged period of time achieves a constant drug level, so that particularly good success can be achieved.

Another possibility is to use estrogens and/or androgens as the medicinally relevant active ingredient. Estrogens can be used, for example, for the permanent treatment of postmenopausal estrogen deficiency syndromes or also for the permanent treatment of male-to-female transsexuality. Androgens can be used for the life-long permanent treatment of female-to-male transsexuality.

In order to facilitate the constant delivery of the active ingredient, it is advantageous if the biodegradable polymers are so chosen that constant degradation is achieved over the entire resorption rate. There are suitable for this purpose the polymers already mentioned, such as polylactides and polyglycoids or polycaprolactans and oxalate-crosslinked oligocaprolactans.

The invention relates further to an implant according to the invention for use in pharmacological permanent treatment, and preferably to an implant according to the invention for use in the treatment of androgenetic hair loss, of postmenopausal estrogen deficiency syndromes, of male-to-female transsexuality and/or of female-to-male transsexuality.

In summary, the invention is also suitable for permanent treatment with any other active ingredient which can permanently be topically released locally in a histocompatible manner. The invention is therefore advantageous in situations which require frequently life-long permanent pharmacological treatment.

If, for example, the implant according to the invention is used with finasteride and/or dutasteride as the active ingredient, the enzyme 5-α-reductase is continuously blocked, so that damaging levels of dihydrotestosterone, which terminally differentiates the hair follicle, can no longer form. A daily intake of finasteride or dutasteride which is not consistently regular, as is the case with onerous daily oral or topical administration, would mean that there would always be an increase, in phases, in the local dihydrotestosterone (DHT) concentrations, with the negative effects on the terminally differentiating hair follicle. This would have the result that the apoptosis program of the hair follicle cells, and thus shriveling of the hair follicle and the development of alopecia, would be encouraged. Accordingly, the use of the implant according to the invention not only improves compliance but also leads to optimization of the efficiency of the therapy itself by ensuring constant drug levels of the relevant active ingredients.

Furthermore, a further problem of conventional therapy is solved, since the possibility of contact of the substances introduced in the implant with persons in the vicinity of the implant wearer is ruled out.

With the implant according to the invention, or the use thereof, continuous and safe administration of active ingredients is made possible, without unnecessarily increasing the outlay in terms of implantation, and better acceptance is achieved.

1.-12. (canceled)

13. An implant for subcutaneous implantation,
having a substantially cylindrical, conical or spiral-shaped base body which has a lateral surface,
wherein the base body consists of a completely resorbable biodegradable polymer,
wherein the implant is configured for blunt implantation,
wherein there are provided on the lateral surface of the base body, in the case of a cylindrical or conical base body, protruding wings which are integrally formed with the base body,
wherein the biodegradable polymer is enriched with a medicinally relevant active ingredient.

14. The implant according to claim 1,
characterized in that
the wings are arranged circumferentially at the entire periphery of the lateral surface, in that the wings, in a radial cross-section of the implant, have a surface substantially similar to a triangle.

15. The implant according to claim 1,
characterized in that
the wings are configured in the manner of a spiral, whereby they form at least one screw thread.

16. The implant according to claim 1,
characterized in that
the implant is configured to be self-inserting and/or self-tapping on rotation about the longitudinal axis of the base body.

17. The implant according to claim 1,
characterized in that
it has a resorption rate of at least three months.

18. The implant according to claim 1,
characterized in that
it has a blunt or rounded tip.

19. The implant according to claims 1,
characterized in that
the medicinally relevant active ingredient is a competitive inhibitor of type 1 and/or type 2 5-α-reductase, in particular dutasteride, finasteride and/or minoxidil.

20. The implant according to claim 1,
characterized in that
the medicinally relevant active ingredient is estrogens and/or androgens.

21. The implant according to claim 1,
characterized in that
the biodegradable polymers have a constant degradation over time.

22. The implant according to claim 1,
characterized in that
there are used as biodegradable polymers polylactide and polyglycoid or polycaprolactan and oxalate-crosslinked oligocaprolactones, wherein the resorption time can be adjusted by the ratio of the two polymers to one another.

23. The implant according to claim 1 for use in pharmacological permanent treatment.

24. The implant according to claim 11 for use in the treatment of androgenetic hair loss, of postmenopausal estrogen deficiency syndromes, of male-to-female transsexuality and/or of female-to-male transsexuality.
